(19) United States
(12) Patent Application Publication
(10) Pub. No.: US 2007/0160559 A1
(43) Pub. Date: Jul. 12, 2007

(54) Skin disinfectant composition and methods for using

(76) Inventor: James A. Roszell, Henderson, NV (US)

Correspondence Address: MERCHANT & GOULD PC, P.O. BOX 2903, MINNEAPOLIS, MN, 55402-0903, US

(21) Appl. No.: 11/331,410

(22) Filed: Jan. 12, 2006

(51) Int. Cl. A61K 8/81; A61K 31/155

(52) U.S. Cl. 424/70.15; 514/634

(57) ABSTRACT

A skin disinfectant composition is provided according to the invention. The skin disinfectant composition comprises an effective amount of a disinfectant active component to provide disinfectant properties to skin tissue, an effective amount of a skin bonding polymer component to hold the disinfectant active component to skin tissue so that the disinfectant active component becomes available on skin tissue to provide disinfectant properties, and water. A method of using a skin disinfectant composition is provided.

### FIELD OF THE INVENTION

The invention relates to a skin disinfectant composition and to methods for using a skin disinfectant composition. In particular, the skin disinfectant composition can include an effective amount of a disinfectant active component and an effective amount of a skin bonding polymer component to hold the disinfectant active component to skin tissue.

### BACKGROUND OF THE INVENTION

Compositions have been developed for enhancing the cutaneous penetration of topically or transdermally delivered pharmacologically active agents. For example, see U.S. Pat. Nos. 5,045,317; 5,051,260; and 4,971,800.

Chlorhexidine gluconate is an antiseptic used as an active ingredient in dental and non-dental applications. Dental products that contain chlorhexidine gluconate are available under the names PERIDEX, PERIOCHIP, and PERIOGARD ORAL RINSE. A non-dental product that includes chlorhexidine gluconate is available under the name HIBICLENS from Regent Medical. The HIBICLENS product is available for general skin cleansing as a surgical scrub, and as a pre-operative skin preparation. The HIBICLENS product is aqueous and contains 4.0 wt. % chlorhexidine gluconate and 4.0 wt. % isopropanol.

Chlorhexidine digluconate is available in a teat dip for use in the dairy industry to prevent mastitis. An exemplary teat dip product contains 0.5 wt. % chlorhexidine digluconate.

There is a general desire to provide products that reduce irritation to skin, chapping and redness of skin, and that provide antimicrobial activity over a relatively long period of time.

### SUMMARY OF THE INVENTION

A skin disinfectant composition is provided according to the invention. The skin disinfectant composition comprises an effective amount of a disinfectant active component to provide disinfectant properties to skin tissue, an effective amount of a skin bonding polymer component to hold the disinfectant active component to skin tissue so that the disinfectant active component becomes available on skin tissue to provide disinfectant properties, and water.

A method of using a skin disinfectant composition is provided according to the invention. The method includes applying the skin disinfectant composition to skin tissue. The skin tissue can include a person's hands.

### BRIEF DESCRIPTION OF THE DRAWING

FIG. 1 is a graph showing the result of Example 1.

### DETAILED DESCRIPTION OF THE INVENTION

A skin disinfectant composition is provided that exhibits prolonged disinfectant properties. In general, disinfectant properties refer to antimicrobial activity, antibacterial activity, antiviral activity, antifungal activity, or a mixture thereof. Prolonged disinfectant properties refers to disinfectant properties that persist over a period of time. In general, the persistence can be considered sufficient so that the skin tissue having the skin disinfectant composition applied thereto can exhibit disinfectant properties for at least two hours after application of the composition to the skin tissue and taking into account washing the skin tissue every half hour for 30 seconds with a mild soap. The skin disinfectant composition preferably provides disinfectant properties at least about four hours after application to skin tissue and taking into account washing the skin tissue every 30 minutes for 30 seconds with a mild soap. The disinfectant composition can be characterized as a composition that resists washing from skin tissue using mild soap.

The skin disinfectant composition can be applied to skin tissue on virtually any part of the body to provide disinfectant properties. One area of the body often in need of disinfectant is the hands. When the skin disinfectant composition is available for application to the hands to provide disinfectant properties, the skin disinfectant composition can be referred to as a hand disinfectant composition.

The skin disinfectant composition can be provided in the form of a lotion and applied to skin tissue by rubbing the composition onto the skin tissue. The skin disinfectant composition can have a viscosity that allows it to be applied to skin tissue conveniently as a lotion. The skin disinfectant composition can have a viscosity that is sufficiently high so that the lotion can be applied from a container (e.g., a tube or a bottle) to a person's hand or a location on the person's body, and the lotion can be rubbed onto the skin tissue. When provided as a lotion, the hand disinfectant composition can have a viscosity of greater than about 3,000 cSt (centistokes). When provided as a lotion, the skin disinfectant composition can be referred to as a skin disinfectant lotion or more simply as a lotion. The skin disinfectant composition can be provided in a form having a viscosity of less than about 3,000 cSt. When the skin disinfectant composition is provided having a viscosity of less than about 3,000 cSt, the skin disinfectant composition can be called a skin disinfectant liquid or more simply as a liquid.

The skin disinfectant composition includes an antimicrobial component, a skin bonding polymer component, and water. Additional components that can be included in the skin disinfecting composition include surfactant, pH modifying agent, coloring agent, preservative, thickening agent, emollient, humectant, antioxidant, fragrance, and chelating agent. The skin disinfecting composition can include any one or more of these additional components.

The skin disinfectant composition can be provided as an emulsion. Exemplary types of emulsions include oil in water emulsions, and water in oil in water emulsions.

The skin disinfectant composition can be provided so that the skin disinfectant composition exhibits at least a 2 log reduction Gram negative bacteria in 15 minutes. Preferably, the skin disinfectant composition can be provided so that it exhibits at least a 3 log reduction against Gram negative bacteria in 15 minutes.

Skin Bonding Polymer Component

The hand disinfectant composition can include a skin bonding polymer component. The skin bonding polymer component can include any polymer that, when applied to the skin, helps hold the disinfectant active component to the skin. The skin bonding polymer component holds the disinfectant active component to the skin tissue for a sufficient length of time to provide a desired disinfectant property. The skin bonding polymer component can be referred to as the polymer component. The polymer component can be characterized as a polymer having an average molecular weight of at least about 2,000, and as a polymer having an average molecular weight of less than about 500,000.

The polymer component can include a hydrophobic polymer/hydrophilic polymer adduct and can include other components. Polymer components that can be used according to the invention include the topical compositions disclosed in U.S. Pat. No. 6,756,059. The entire disclosure of U.S. Pat. No. 6,756,059 is incorporated herein by reference.

The skin disinfectant composition can bind or adhere to skin tissue for a length of time, and can hold or contain the disinfectant active component within the composition. It is expected that the skin disinfecting composition is able to adhere or bind to skin tissue for at least about two hours and preferably at least about four hours and hold the disinfectant active component contained therein in proximity to skin tissue for that length of time. In general, it is expected that the skin disinfecting composition will adhere the disinfectant active component to skin tissue for a length of time sufficient to provide desired disinfectant properties. It is believed that a person can shower or wash relatively soon after application of the skin disinfecting composition, and the polymer component will hold the disinfectant active component to the skin. In general, after application of the skin disinfectant composition to skin tissue, the skin disinfectant composition can provide desired disinfectant properties for at least about two hours, and preferably at least about four hours, after application of the skin disinfectant composition and after allowing for washing the skin tissue every 30 minutes for 30 seconds using a mild soap. A mild soap includes, for example, those soaps available under the names DOVE and SOFT SOAP.

The polymer component can be prepared from a topical composition precursor. The topical composition precursor can be prepared by melt processing a hydrophobic polymer composition and a hydrophilic polymer composition to provide an interaction between the hydrophobic polymer composition and the hydrophilic polymer composition. It should be understood that the phrase “melt processing” refers to mixing the hydrophobic polymer composition and the hydrophilic polymer composition under conditions that provide that the hydrophobic polymer component of the hydrophobic polymer composition and the hydrophilic polymer component of the hydrophilic polymer composition are in a liquid state so that they sufficiently mix. When the polymers are sufficiently mixed, it is believed that an interaction forms between the hydrophobic polymer component and the hydrophilic polymer component. The melt processing temperature can be at least about 50° C. and can be at least about 70° C. to generate this interaction.

It is believed the interaction exhibited between the hydrophobic polymer component and the hydrophilic polymer component is a type of complex formation reaction, and that the complex, once formed, can be stable in water at temperatures up to 65° C. and at a pH range of 3.0 to 9.0. By stable, it is meant that the complex does not favor disassociation under these conditions. It is believed that this interaction provides the skin disinfectant composition with an ability to bind or hold onto the disinfectant active component that may be hydrophobic or relatively water insoluble, allows the skin disinfectant composition to be emulsified in water, and provides the skin disinfectant composition with an ability to bind to skin. The result of the interaction between the hydrophobic polymer component and the hydrophilic polymer component can be referred to as a hydrophobic polymer/hydrophilic polymer adduct. It should be understood that the term “adduct” is used to refer to the interaction between the hydrophobic polymer component and the hydrophilic polymer component. The interaction may be a form of complexing, but that is only theory. Accordingly, it should be understood that the term “adduct” is not meant to limit the polymer component to a particular theory of interaction.

It is believed that the interaction between the hydrophobic polymer component and the hydrophilic polymer component can be achieved more easily in the absence of water. It is expected that that if the hydrophilic polymer component becomes dissolved in water before forming the complex, it can be more difficult to sufficiently mix the hydrophobic polymer component and the hydrophilic polymer component to provide the desired level of interaction. Although a convenient technique for providing the desired level of interaction between the hydrophobic polymer component and the hydrophilic polymer component is melt mixing, it is expected that other techniques can be used to achieve the desired level of interaction. For example, it may be possible to use a nonaqueous solvent to help achieve the desired level of interaction.

The hydrophobic polymer composition that can be used according to the invention includes at least one hydrophobic polymer and can include a mixture of hydrophobic polymers. The hydrophobic polymer composition can include components having repeating pyrrolidone/alkylene groups. Exemplary polymers having repeating pyrrolidone/alkylene groups include poly(vinylpyrrolidone/alkylene)polymers.

Poly(vinylpyrrolidone/alkylene)polymers include those polymers obtained by polymerizing alkylene substituted vinylpyrrolidone. Poly(vinylpyrrolidone/alkylene)polymers can be represented by the following general formula: 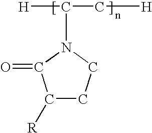
wherein R represents a carbon chain substitute such as an alkylene group and n represents the number of repeating units. The R group is preferably sufficiently long so that the polymer remains relatively water insoluble and should not be too long so that the polymer is difficult to melt process. The alkylene group can contain a length of at least about 10 carbon atoms and can contain less than about 30 carbon atoms. The alkylene group can contain about 14 carbon atoms to about 22 carbon atoms, and can contain about 15 carbon atoms to about 19 carbon atoms.

The poly(vinylpyrrolidone/alkylene)polymers that can be used according to the invention can have a molecular weight that is sufficiently high so that the polymer maintains its water insolubility but the molecular weight should not be so high that it becomes difficult to melt process the polymer. The weight average molecular weight of the poly(vinylpyrrolidone/alkylene)polymer can be between about 3,000 and about 400,000. Another way to characterize the size of the poly(vinylpyrrolidone/alkylene)polymer is by the number of repeating units (n). In the case of a poly(vinylpyrrolidone/alkylene)polymer having a weight average molecular weight of about 6,000 to about 30,000, the poly(vinylpyrrolidone/alkylene)polymer can have about 20 to about 80 repeating units, and can have about 30 to about 50 repeating units. It should be understood that repeating units refer to the residues of vinylpyrrolidone/alkylene groups.

Exemplary poly(vinylpyrrolidone/alkylene)polymers that can be used according to the invention include poly(vinylpyrrolidone/1-eicosene) and poly(vinylpyrrolidone/hexadecene). Poly(vinylpyrrolidone/1-eicosene) can be referred to as PVPE and is commonly used in pharmaceutical and cosmetic preparations. An exemplary form of PVPE for use according to the invention includes about 43 to 44 repeating units in length and has a weight average molecular weight of about 17,000 and can be characterized as a paraffin-like solid. This particular PVPE is highly insoluble in water, and has an extremely low oral toxicity (LD₅₀>17000 mg/kg) and exhibits no demonstrable dermal toxicity. Poly(vinylpyrrolidone/1-hexadecene) can be referred to as PVPH. An exemplary form of PVPH is available as a viscous yellow liquid that is insoluble in water and has a low oral toxicity (LD₅₀>64000 mg/kg), has about 39 to 40 repeating units, a molecular weight of about 14,000, and exhibits no demonstrable dermal toxicity.

PVPE and PVPH differ in the length of the hydrocarbon side chain, and are used extensively in the skin care industry, usually in concentrations of less than 1% by weight, because of their ability to bind to skin. Because the skin care industry generally prefers to apply actives to skin using a water-based composition, the use of PVPE and PVPH often requires solvents, surfactants, and emulsifiers to stabilize these polymers in a water emulsion. However, many of the solvents, surfactants and emulsifiers used to stabilize PVPE and PVPH in a water emulsion lack the low dermal toxicities of PVPE and PVPH. PVPE and PVPH by themselves lack a cosmetically elegant appeal when applied directly to the skin. They tend to be sticky and greasy.

The hydrophobic polymer composition used according to the invention can be provided as a mixture of different poly(vinylpyrrolidone/alkylene)polymers. The mixture of different poly(vinylpyrrolidone/alkylene)polymers can include at least 5 wt. % of a first poly(vinylpyrrolidone/alkylene)polymer based on the weight of the hydrophobic polymer composition. The hydrophobic polymer composition can include about 5 wt. % to about 54 wt. % of the first poly(vinylpyrrolidone/alkylene)polymer. The second poly(vinylpyrrolidone/alkylene)polymer can be provided in an amount of at least about 46 wt. % and can be in a range of about 46 wt. % to 95 wt. % based on the weight of the hydrophobic polymer composition. For a hydrophobic polymer composition containing a first poly(vinylpyrrolidone/alkylene)polymer and a second poly(vinylpyrrolidone/alkylene)polymer, the mole ratio of the first polymer to the second polymer can be about 1:22 to about 1:1. When the hydrophobic polymer composition contains a mixture of different poly(vinylpyrrolidone/alkylene)polymers, the poly(vinylpyrrolidone/alkylene)polymers can be selected to provide improved properties compared to a composition having a hydrophobic polymer composition containing a single poly(vinylpyrrolidone/alkylene)polymer.

When the hydrophobic polymer composition is provided as a mixture of PVPH and PVPE, the PVPH can be provided in a range of about 46 wt. % to about 95 wt. % and the PVPE can be provided in a range of about 5 wt. % to about 65 wt. %, based upon the weight of the hydrophobic polymer composition.

The hydrophilic polymer composition that can be used according to the invention includes at least one hydrophilic polymer and may include a mixture of hydrophilic polymers. The hydrophilic polymers that can be used according to the invention include polymers having repeating carboxylic acid groups, hydroxyl groups, or both carboxylic acid groups and hydroxyl groups. Exemplary hydrophilic polymers that can be used according to the invention include polyacrylic acid polymers, poly(maleic acid/methylvinylether)copolymers, starch, derivatives of starch, polyvinyl alcohol, cellulose, derivatives of cellulous, carboxymethyl cellulous, cyclodextrins, dextrans, or mixtures thereof. The hydrophilic polymers should have a molecular weight that is not too high so that the hydrophilic polymer becomes difficult to process.

Polyacrylic acid polymers that can be used according to the invention include those having a weight average molecular weight of at least about 50,000. Polyacrylic acid polymers that can be used include those having a weight average molecular weight between about 50,000 to about 4,000,000. The polyacrylic acid polymers can have a level of cross-linking that is less than about 1% to help provide hydrophilic properties. A general structural representation of polyacrylic acid polymers is shown below: 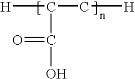
wherein n is the number of repeating units. The number n can be about 1,000 to about 20,000.

Poly(maleic acid/methylvinylether)copolymers that can be used according to the invention can have a weight average molecular weight of at least about 50,000, and can have a weight average molecular weight of about 50,000 to about 4,000,000. The weight average molecular weight can be about 70,000 to 2,500,000. A general structural representation of poly(maleic acid/methylvinylether)copolymers is shown below: 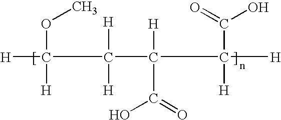
wherein n is the number of repeating units. The number n can be about 200 to about 20,000.

Additional hydrophilic polymers that can be used according to the invention include starch, derivatives of starch, polyvinyl alcohol, cellulose, derivatives of cellulose, carboxymethyl cellulose, cyclodextrins, and dextrans. The weight average molecular weight of the hydrophilic polymers is preferably sufficient to provide solubility in water but not too high to become difficult to process. Exemplary starches include amylopectin and polyglucose. Starches that can be used according to the invention can have a weight average molecular weight of about 50,000 to about 20,000,000. A derivative of starch that can be used according to the invention includes partially hydrolyzed starch. Cellulose that can be used according to the invention can have a weight average molecular weight of about 50,000 to about 15,000,000. Polyglucose that can be used according to the invention can be characterized as low fraction polyglucose having a weight average molecular weight of about 60,000 to about 90,000, and high fraction polyglucose having a weight average molecular weight of about 90,000 to about 300,000. An exemplary low fraction polyglucose material that can be used according to the invention is available under the name Dextran-70. In general, this type of polyglucose has all alpha 1-6 linkages. Starch derivatives that can be used according to the invention include those starch derivatives having alpha 1-4 linkages. An example of this type of starch derivative includes cyclodextrins. Exemplary cyclodextrins that can be used according to the invention include those that act to provide a cavity within the molecule large enough to contain components desirable for topical applications. Cyclodextrins that can be used according to the invention can have a molecular weight of about 900 to about 1,400. Polyvinyl alcohols that can be used according to the invention include those with a weight average molecular weight of about 50,000 to about 200,000.

Exemplary hydrophilic polymers that can be used according to the invention include those polymers having a melting temperature that allows for melt processing without decomposition of the polymer. Exemplary poly(maleic acid/methylvinylether)copolymers that can be used include those having a melting temperature range of about 60° C. to about 65° C. and a maximum temperature range of about 80° C. to about 90° C. The melting temperature refers to the temperature at which the polymer melts, and the maximum temperature refers to the temperature at which the polymer begins to decompose. Exemplary polyacrylic acid polymers that can be used include those having a melting temperature range of about 65° C. to about 70° C. and a maximum temperature range of about 80° C. to about 90° C. Exemplary carboxymethyl cellulose polymers that can be used include those having a melting temperature range of about 55° C. to about 60° C. and a maximum temperature range of about 75° C. to about 80° C. Exemplary polyvinyl alcohol polymers that can be used include those having a melting temperature range of about 50° C. to about 55° C. and a maximum temperature range of about 65° C. to about 70° C. Exemplary starches that can be used include those having a melting temperature range of about 40° C. to about 45° C. and a maximum temperature range of about 50° C. to about 55° C. Exemplary dextrans that can be used include those having a melting temperature range of about 37° C. to about 40° C. and a maximum temperature range of about 45° C. to about 50° C. Exemplary β-cyclodextrins that can be used according to the invention include those having a melting temperature range of about 40° C. to about 45° C. and a maximum temperature range of about 65° C. to about 70° C.

The hydrophobic polymer composition and the hydrophilic polymer composition can be combined and heated to at least about 50° C. to provide a polymer melt. The composition can be heated to at least about 70° C. under mixing to form complexes between the hydrophobic and hydrophilic polymers. It should be understood that a polymer melt refers to a polymer that flows or becomes a liquid when heated and is not meant to refer to a polymer that forms a liquid as a result of being dissolved in a solvent.

The complex formation step can be carried out in a relatively anhydrous environment. That is, the amount of water provided in the composition during the complex formation step can be less than about 1 wt. %. Once the desired level of complex formation has occurred, the composition can be hydrated with water.

The hydrophobic polymer composition and the hydrophilic polymer composition can be mixed together in amounts sufficient to provide a ratio of pyrrolidone groups to the combination of carboxylic acid groups and hydroxyl groups of about 1:1 to about 5:1. The ratio of the structures causing the observed interaction between the hydrophobic polymer composition and the hydrophilic polymer composition can be referred to as “functional group parity.” The ratio of pyrrolidone groups to the combination of carboxylic acid groups and hydroxyl groups can be about 1.5:1 to about 3:1. In order to drive the complex formation reaction, it is desirable to provide an imbalance between the two types of groups. Accordingly, it is generally desirable to provide more of the pyrrolidone groups than the combination of carboxylic groups and the hydroxyl groups. It should be understood that the reference to a “combination of carboxylic groups and hydroxyl groups” refers to the total amount of carboxylic groups and hydroxyl groups present but does not require the presence of both carboxylic groups and hydroxyl groups. For example, the value of the combination of carboxylic groups and hydroxyl groups can be determined for a composition that contains only carboxylic groups. Similarly, the value can be determined for a composition that contains only hydroxyl groups.

During the complex formation step, the amounts of hydrophobic polymer composition and hydrophilic polymer composition can be characterized on a weight percent basis. For example, about 2 wt. % to about 28 wt. % hydrophilic polymer composition and about 72 wt. % to about 98 wt. % hydrophobic polymer composition can be combined to provide for complex formation. About 8 wt. % to about 25 wt. % hydrophilic polymer composition and about 72 wt. % to about 95 wt. % hydrophobic polymer composition can be combined to form the complex. During the complex formation step, the amount of water available in the composition can be less than about 1 wt. %. Although the complex forming composition can be relatively anhydrous, it is expected that the amount of water will be between about 0.3 wt. % and about 1.0 wt. %.

Once the hydrophobic polymers and the hydrophilic polymers have sufficiently reacted or interacted to form a complex, water can be added to the composition to provide a stable aqueous composition that can be relatively easily further hydrated. It has been found that the first hydration of the topical composition precursor is the most difficult hydration step because of the need to control the conditions of hydration. After the first hydration to a water content of at least about 30 wt. %, it is expected that further hydrations to higher water contents are relatively easy and can be accomplished by simply mixing the composition with water. Accordingly, the amount of water provided in the composition when made available as a concentrate for shipment is preferably between about 30 wt. % and about 45 wt. %. When the composition includes about 30 wt. % to about 45 wt. % water, it is expected that the composition can include about 3 wt. % to about 10 wt. % hydrophilic polymer composition and about 30 wt. % to about 50 wt. % hydrophobic polymer composition.

Water can be added to the relatively anhydrous composition by mixing water and the relatively anhydrous composition at a temperature and for a time sufficient to allow the composition to become hydrated without losing significant amounts of interaction between the hydrophobic polymer composition and the hydrophilic polymer composition. The relatively anhydrous composition can be hydrated by heating to at least 60° C. and adding water while mixing. The composition can be heated to at least about 65° C. and to at least about 70° C. An exemplary temperature range is about 65° C. to about 80° C.

The relatively anhydrous composition can be referred to as the topical composition precursor and generally refers to the hydrophobic polymer/hydrophilic polymer adduct. The polymer component for the hand disinfecting composition can refer to a composition that contains only the hydrophobic polymer/hydrophilic polymer adduct, and it can refer to a composition wherein the hydrophobic polymer/hydrophilic polymer adduct is diluted with water. In general, it is desirable to have a sufficient amount of water in the polymer component that allows one to formulate the polymer component into the skin disinfecting composition according to the invention. If there is too little water in the polymer component, it may become difficult to formulate the skin disinfecting composition. For example, the polymer component can contain water in an amount of up to about 95 wt. %. The polymer component can have a water concentration of about 30 wt. % to about 45 wt. %.

Additional components can be added to the skin bonding polymer composition. For example, it may be desirable to add a component that helps stabilize the hydrophobic polymer/hydrophilic polymer adduct, and to help preserve and/or maintain the composition.

Disinfectant Active Component

The skin disinfectant composition includes a disinfectant active component. The skin disinfectant composition can include any disinfectant active component that is compatible with the skin bonding polymer component and provides desired disinfectant properties. In general, compatibility of the disinfectant active component with the skin bonding polymer component refers to the lack of phase separation between the disinfectant active component and the skin bonding polymer component. It is generally desirable for the skin bonding polymer component to hold the disinfectant active component to skin tissue to provide the desired disinfectant properties for a desired length of time.

Chlorhexidine can be referred to as 1,6-di(4-chlorophenyl-diguanido)hexane. In general, chlorhexidine is a strong base and is practically insoluble in water (0.008% wt/vol. at 20° C.). To make chlorhexidine soluble in water, it is typically reacted with acids to form salts of the RX₂ type. See Denton, Disinfection, Sterilization and Preservation, Fifth Edition, Chapter 15, pages 321-336, Lippincott, Williams and Wilkins, Dec. 15, 2000. It should be understood that the reference to “% wt/vol. at 20° C.” refers to the weight percent of the component that is soluble in 100 ml water at 20° C.

Chlorhexidine digluconate is a commonly used antiseptic. Because of its relatively high water solubility, chlorhexidine digluconate has a tendency to phase separate from the skin bonding polymer component. Accordingly, chlorhexidine can be modified to make it more compatible with the skin bonding polymer component. In general, the modification can provide a modified chlorhexidine that is more compatible with the skin bonding polymer component so that the skin bonding polymer component holds the modified chlorhexidine to skin tissue. The chlorhexidine modified to be more compatible with the skin bonding polymer component can be referred to as modified chlorhexidine. The modified chlorhexidine can be provided as a chlorhexidine salt.

Chlorhexidine can be provided as a chlorhexidine salt having a water solubility of less than about 1% wt/vol in water at 20° C. by selecting the acid that reacts with the chlorhexidine to provide a more neutralized chlorhexidine salt having the desired level of low water solubility. In general, it is desirable to neutralize the chlorhexidine so that the chlorhexidine does not harm the skin tissue. Furthermore, it is desirable to provide the chlorhexidine salt as a relatively water insoluble salt so that the skin bonding polymer component can hold the chlorhexidine salt while the polymer component is bonded to the skin tissue. Because the polymer component can hold the chlorhexidine salt while the polymer component is bonded to the skin tissue, the chlorhexidine salt can be made available to provide desired disinfectant properties while the polymer component remains bonded to the skin tissue.

Exemplary acids that can react with chlorhexidine to provide a chlorhexidine salt having a relatively low water solubility include fatty acids, polycarboxylic acids, mineral acids, and mixtures thereof. Exemplary fatty acids include those fatty acids having a fatty chain that helps reduce water solubility. In general, the fatty chain can have at least about eight carbon atoms. The fatty chain should not be so large that the fatty acid is not capable of sufficiently reacting with the chlorhexidine. For example, it may be desirable to provide the fatty chain having less than about 30 carbon atoms. Furthermore, the fatty chain can be characterized as saturated or unsaturated. Exemplary fatty acids that can be used include stearic acid, oleic acid, and mixtures of stearic acid and oleic acid. The polycarboxylic acid can be used to provide cross linking between chlorhexidine molecules to decrease water solubility. Cross linking can create an increased molecular weight that can provide reduced water solubility. Exemplary polycarboxylic acids include dicarboxylic acids. The polycarboxylic acids, such as dicarboxylic acids, can be characterized as aliphatic, aromatic, or cyclic. In order to provide decreased water solubility, the polycarboxylic acids can be preferably provided so that they do not internally form a salt with the chlorhexidine by forming a cyclic molecule. For example, it may be desirable to avoid having a dicarboxylic acid react with both ends of the chlorhexidine molecule to form a cyclic salt. Accordingly, the number of carbon atoms between carboxylic acid groups can be about 1 to about 12. An exemplary dicarboxylic acid includes succinic acid. Mineral acids can be provided to react with the chlorhexidine to reduce water solubility. Exemplary mineral acids include hydrochloric acid, hydrobromic acid, nitric acid, sulfuric acid, carbonic acid, phosphoric acid, or mixtures thereof.

The chlorhexidine salt that can be used can be characterized as relatively water insoluble. In general, a relatively water insoluble chlorhexidine salt can be characterized as a salt having a water solubility of less that about 1% wt/vol. in water at 20° C., preferably less that about 0.5% wt/vol. in water at 20° C., and more preferably less than about 0.1% wt/vol. in water at 20° C.

The chlorhexidine salt can be prepared by mixing chlorhexidine with an acid in water at a temperature sufficient to enhance the salt forming reaction. The temperature of the composition can be heated to about 45° C. to about 50° C. to enhance the salt formation. In general, as the chlorhexidine salt forms, it tends to precipitate out and form a slurry. Although basic chlorhexidine can be provided as a starting material, it should be appreciated that other salts of chlorhexidine can be provided as a starting material. For example, chlorhexidine gluconate can be used as a starting material.

Exemplary forms of chlorhexidine salt include stearic acid salts of chlorhexidine(chlorhexidine stearate), succinate acid salts of chlorhexidine(chlorhexidine succinate), and mixtures of chlorhexidine stearate and chlorhexidine succinate. A desired release of chlorhexidine from the skin disinfectant composition can be found when a mixture of chlorhexidine stearate and chlorhexidine succinate is used. When a mixture of chlorhexidine stearate and chlorhexidine succinate is used, the mixture can be provided at a weight ratio of chlorhexidine stearate to chlorhexidine succinate of about 1:10 to about 10:1, and preferably 1:5 to about 5:1.

The skin disinfectant composition can include disinfectant active component in an amount sufficient to provide desired disinfectant properties. Exemplary ranges of the disinfectant active component in the skin disinfectant composition include about 0.1 wt. % to about 5 wt. %, about 0.25 wt. % to about 4 wt. %, and about 0.5 wt. % to about 3 wt. %.

An advantage of the skin disinfectant composition that has been observed is the release of chlorhexidine as a result of hand washing. That is, after a person has applied the skin disinfectant composition to his or her hands, a subsequent washing of the hands tends to release chlorhexidine so that the chlorhexidine is available to provide disinfectant properties on the skin tissue. The release of chlorhexidine as result of washing of hands can be observed during the useful life of the skin disinfectant composition after application to the hands.

Water

The skin disinfectant composition can include water in an amount sufficient to allow the composition to be applied to skin tissue while providing the desired coverage over the skin tissue. The water component can be provided as deionized water, filtered water, distilled water, reverse osmosis water, or tap water. In the event that the water includes hardness or other components, it may be desirable to include builders, sequestrants, and chelating agents to handle the water hardness. In general, the hand disinfectant composition can include at least about 50 wt. % water. In addition, it is expected that if there is too much water, the emulsion might become unstable. In general, the amount of water in the skin disinfectant composition can be less than about 95 wt. %. The amount of water in the skin disinfectant composition can be about 65 wt. % to about 93 wt. %.

Surfactant Component

The skin disinfectant composition can include a surfactant component to help maintain the composition as an emulsion. In general, an emulsion refers to a composition that resists phase separation after sitting at room temperature for a couple of months. In general, it is expected that the skin disinfectant composition can be stored in a warehouse or in a storage closet for at least two months and can remain as an emulsion during that two month period. Preferably, the skin disinfectant composition can remain as an emulsion for at least one year or at least two years. The ability of the skin disinfectant composition to remain as an emulsion can be tested according to an accelerated stability test where the composition is held at 45° C. for two months. It is expected that this accelerated stability test for two months roughly corresponds to a period of about two years at room temperature. In general, it is expected that the hand disinfecting composition can remain as an emulsion after sitting for one month at 45° C. and preferably at least two months at 45° C.

Exemplary surfactants that can be used as the surfactant component include nonionic surfactants that help stabilize the emulsion and provide a generally even distribution of the disinfectant active component. Exemplary nonionic surfactants that can be used include glycerol stearate such as glycerol monostearate, polysorbate such as that available under the name Tween 60, and polyoxyethylene stearate. In addition, mixtures of nonionic surfactants can be included including mixtures of polysorbate and glycerol stearate. An additional nonionic surfactant that can be used includes an ethoxy surfactant, a propoxy surfactant, or an ethoxy/propoxy surfactant. An exemplary ethoxy/propoxy surfactant includes a 10 carbon chain and 9 PO/EO surfactant available under the name Lutensol XP-90 from BASF.

It is believed that anionic surfactants may be useful as part of the surfactant component. In general, it is expected that anionic surfactants have a greater tendency to cause irritation to skin tissue.

The skin disinfectant composition can include an amount of surfactant component sufficient to provide the composition with a desired emulsion stability and sufficiently low viscosity without foaming. The amount of the surfactant component in the hand disinfecting composition, can be about 0.5 wt. % to about 6 wt. %, and can be about 1 wt. % to about 5 wt. %. It should be understood that the skin disinfectant composition can be provided without any surfactant component.

pH Adjusting Agent

The hand disinfecting composition can include a pH adjusting agent to provide the hand disinfecting composition with a pH that helps stabilize the disinfectant active component. Exemplary pH adjusting agents that can be used include citric acid, lactic acid, acetic acid, propionic acid, and mixtures thereof.

The polymer component of the lotion may be at least in part responsible for reducing the irritability of the skin disinfectant composition at low pH values. For example, it is believed that the polymer component may help reduce irritation of skin tissue. The skin disinfectant composition can be provided without any pH modifier, if desired.

Thickener

Thickeners that can be incorporated into the skin disinfectant composition include those components that thicken or increase the viscosity of the skin disinfectant composition so that the skin disinfectant composition can be readily applied to skin. Thickeners that can be used in the skin disinfectant composition include those components often referred to as viscosity controlling agents.

Exemplary thickeners or viscosity controlling agents that can be provided in the hand disinfecting composition include cellulose gum, alkane triols; acrylates; substituted celluloses such as hydroxy ethyl cellulose, methylcellulose, and hydroxypropyl cellulose; cetyl alcohol; gums such as natural gums or synthetic gums; long chain alcohols such as those having about 9 to about 24 carbon atoms; polyglycols such as polyethylene glycols, polypropylene glycols, polybutylene glycols, polyethylene propylene glycols, or mixtures thereof; waxes such as natural waxes or synthetic waxes; hydrogenated oils; glycol esters; fatty acid esters; long chain acids; acid amides; silicates; and mixtures thereof. An exemplary thickener that can be used is hydroxyethyl cellulose.

The skin disinfectant composition may or may not include a thickener. When the skin disinfectant composition includes a thickener, the thickener can be provided in an amount that provides the desired level of thickening. The hand disinfecting composition can include a thickener in an amount of least about 0.1 wt. % and can include a thickener in an amount of at least about 0.4 wt. %. In addition, the thickener can be provided in an amount of less than about 2 wt. %, and can be provided in an amount of less than about 1.0 wt. %. The skin disinfectant composition can be provided without any thickener, if desired.

Emollient

The skin disinfectant composition can include an emollient for improving the texture of the composition. An emollient is an oleaginous or oily substance which helps to smooth and soften the skin, and may also reduce its roughness, cracking or irritation. Exemplary suitable emollients include mineral oil, having a viscosity in the range of 50 to 500 centipoise (cps), lanolin oil, coconut oil, cocoa butter, olive oil, almond oil, macadamia nut oil, synthetic jojoba oils, natural sonora jojoba oils, safflower oil, corn oil, liquid lanolin, aloe vera, cottonseed oil, and peanut oil.

Other suitable emollients include squalane, castor oil, polybutene, odorless mineral spirits, sweet almond oil, avocado oil, clophyllum oil, ricin oil, vitamin E acetate, olive oil, linolenic alcohol, oleyl alcohol, the oil of cereal germs such as the oil of wheat germ, isopropyl palmitate, octyl palmitate which is commercially available as Lexol EHP, tradename of Inolex Co. of Philadelphia, Pa., isopropyl myristate, hexadecyl stearate, butyl stearate, decyl oleate, acetyl glycerides, the octanoates and benzoates of (C₁₂-C₁₅) alcohols, the octanoates and decanoates of alcohols and polyalcohols such as those of glycol and glycerol, ricin oleates of alcohols and poly alcohols such as those of isopropyl adipate, hexyl laurate and octyl dodecanoate.

Other suitable emollients which are solids or semi-solids at room or ambient temperatures may be used in amounts sufficient to provide liquid topical compositions. Such solid or semi-solid cosmetic emollients include hydrogenated lanolin, hydroxylated lanolin, acetylated lanolin, petrolatum, isopropyl lanolate, butyl myristate, cetyl myristate, myristyl myrislate, myristyl lactate, cetyl alcohol, isostearyl alcohol and isocetyl lanolate. Exemplary emollients include stearic acid, cetyl alcohol, natural and synthetic esters such as coconut oil.

The hand disinfecting composition can include the emollient in an amount sufficient to provide a silky feel. An exemplary range of the emollient in the hand disinfecting composition can be at least about 0.5 wt. %. In addition, the hand disinfecting composition can include an emollient in an amount of less than about 3 wt. %. It should be understood that the emollient is an optional component of the skin disinfectant composition. The skin disinfectant composition can be provided without an emollient, if desired.

Moisturizer

The skin disinfectant composition can include a moisturizer to provide a desired moisturizing effect to skin tissue. The moisturizer can be provided as a humectant. In general, a humectant is a moistening agent that promotes retention of water due to its hydroscopic properties. Exemplary humectants include glycerine, polymeric glycols such as polyethylene glycol and polypropylene glycol, and sorbitols such as sorbitol solution, pyrrolidone carboxylic acid, urea, or mixtures thereof. The hand disinfecting composition can be provided without a moisturizer.

When the skin disinfectant composition includes a moisturizer, it can be included in an amount of at least about 0.5 wt. %. In addition, the skin disinfectant composition can include a moisturizer in an amount of less than about 5 wt. %. It should be understood that the skin disinfectant composition can be provided without a moisturizer.

Preservatives

The skin disinfectant composition can include preservatives for prevention of bacterial, fungal, and/or yeast contamination. Exemplary preservatives that can be used in the hand disinfecting composition include phenoxyethanol, benzoic acid, derivatives and salts of benzoic acid, parabens, oxazolidines, chlorinated aromatic compounds and phenols, hydantoins, cresols and derivatives, imiazolindinyl urea, lodopropanol butylcarbamate, sulfites, and bisulfites. The skin disinfectant composition can include any of the preservatives commonly used or known to be suitable for topically applied compositions.

The skin disinfectant composition can be formulated without a preservative. It is expected that the preservative will increase the shelf life of the skin disinfectant composition by reducing or preventing the growth of bacteria, fungus, and/or yeast. When the skin disinfectant composition includes a preservative, the preservative is preferably provided in an amount sufficient to provide a desired level of protection from growth of bacteria, fungus, and/or yeast.

In general, for most preservatives, it is expected that the amount of preservative can be provided at a level of about 0.1 wt. % to about 1.0 wt. %, and can be provided at a level of about 0.2 wt. % to about 0.5 wt. %, based on the weight of the skin disinfectant composition. Because of the relatively large amount of the disinfectant active component in the skin disinfectant composition, the skin disinfectant composition can be provided without a supplemental preservative.

Antioxidants

The hand disinfecting composition can include antioxidants to help increase the shelf life of the skin disinfectant composition. Exemplary antioxidants that can be used include vitamins such as vitamin E, vitamin E acetate, vitamin C, vitamin A, and vitamin D, and derivatives thereof. Exemplary antioxidants include α-tocopherols which can be characterized as natural or synthetic Vitamin E. Additional exemplary antioxidants include propyl, octyl and dodecyl esters of gallic acid, butylated hydroxyanisole (BHA) (usually as a mixture of ortho and meta isomers), butylated hydroxytoluene (BHT), and nordihydroguaiaretic acid, and alkylated parabens such as methylparaben and propylparaben. Another type of antioxidant includes a reducing component such as a reducing sugar to stabilize the disinfectant active component. And exemplary reducing sugar includes glucose.

The skin disinfectant composition can be formulated without an antioxidant. When the hand disinfecting composition includes an antioxidant, the antioxidant can be provided in an amount that provides antioxidant properties in the skin disinfectant composition. In general, it is expect that the antioxidant can be provided in an amount of about 0.2 wt. % to about 2 wt. %, and can be provided in an amount of about 0.7 wt. % to about 1.5 wt. %, based on the weight of the hand disinfecting composition. In the case of vitamin E, it is expected that the vitamin E can be included in the skin disinfectant composition in an amount of about 0.1 wt. % to about 1 wt. %, and can be included in an amount of about 0.3 wt. % to about 0.8 wt. %. It should be appreciated that the hand disinfecting composition can be provided without an antioxidant.

Chelating Agents

Chelating agents are substances used to chelate or bind metallic ions with a certain heterocyclic ring structure so that the ion is held by chemical bonds from each of the participating rings. Suitable chelating agents include ethylene diaminetetraacetic acid (EDTA), EDTA trisodium, EDTA tetrasodium, calcium disodium edetate, EDTA trisodium, EDTA tetrasodium and EDTA dipotassium. One or more chelating agents can optionally be included in the emulsion in amounts ranging from about 0.001 to about 0.1 weight percent. It should be appreciated that the skin disinfectant composition can be provided without a chelating agent.

Fragrances

Fragrances are aromatic compounds which can impart an aesthetically pleasing aroma to the skin disinfectant composition. Typical fragrances include aromatic materials extracted from botanical sources (i.e. rose petals, gardenia blossoms, jasmine flowers, etc.) which can be used alone or in any combination to create essential oils. Alternatively, alcoholic extracts may be prepared for compounding fragrances. One or more fragrances can optionally be included in the composition in an amount ranging from about 0.001 to about 10 weight percent, preferably about 0.05 to about 5 percent. It should be appreciated that the skin disinfectant composition can be provided without a fragrance.

### EXAMPLE 1

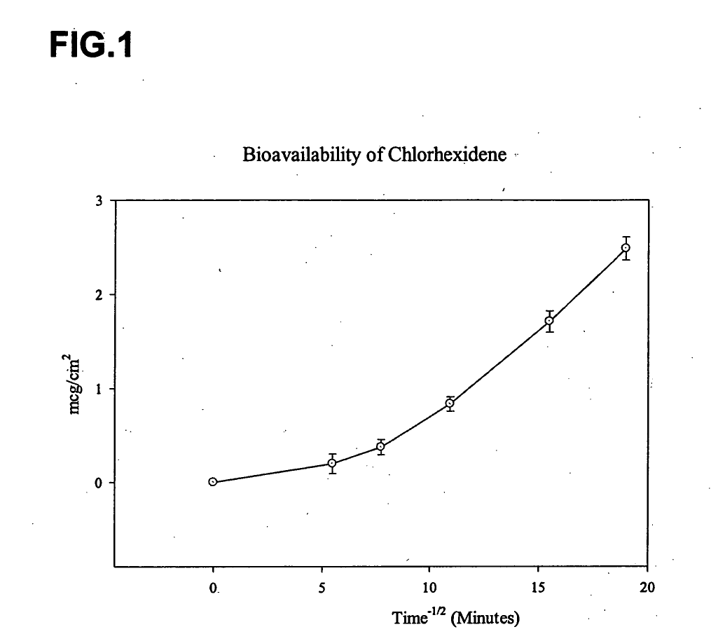

Results of a bioavailability test in Franz Cells for the composition shown in Table 1 are reported in FIG. 1.

The data clearly shows an accelerating sustained release of active ingredient over a 6 hour time. This formulation has also shown 3-6 log reductions in a variety of disease causing bacteria, and 2 log reductions in H5N1 Avian Flu virus. This formulation demonstrates a 2.5-3 year shelf-life in accelerated stability testing.

TABLE 1

| Component | Gms/L | % by wt. |
|---|---|---|
| Water | 853.00 | 85.300 |
| Hydroxy Ethyl Cellulose | 8.00 | 0.80 |
| Glucose | 22.50 | 2.25 |
| Chlorhexidine | 22.50 | 2.250 |
| Nonionic Surfactant* | 8.00 | 0.80 |
| Skin Bonding Polymer | 50.00 | 5.00 |
| Component** | | |
| Stearic Acid | 10.00 | 1.00 |
| Succinic Acid | 9.00 | 0.90 |
| Aloe Vera | 2.00 | 0.20 |
| Coconut Oil | 10.00 | 1.00 |
| Pyrrolidone Carboxylic | 5.00 | 0.50 |
| Acid*** | | |

pH = 4.65
*The nonionic surfactant used was a nonionic surfactant having a 10 carbon atom chain and 9 PO/EO groups and is available under the name Lutensol XP-90 from BASF.
**A result of 4 wt. % carboxymethyl cellulose, 45 wt. % poly(vinylpyrrolidone/1-hexadecene), 5 wt. % poly(vinylpyrrolidone/eicosene), and 45.5 wt. % water.
***Pyrrolidone carboxylic acid is available as PCA-50.

### EXAMPLE 2

The primary objective was to determine the virucidal activity of the composition of Table 1 against NIBRG-14 (H5N1) virus at one concentration for four incubation time points.

The secondary objective was to assess any cytotoxic potential of the test article on the MDCK cell line.

In the cytotoxicity assay, the composition was tested for toxicity at a 100% v/v concentration.

In the virucidal assay, the composition underwent a 9/10 dilution when it was mixed with the virus (see section 8.4). Therefore, the concentration tested in the virucidal assay was 90% v/v.

Control Reference Articles

The controls utilized in the toxicity assay are:
Cell only control: untreated cell. This was a negative control for tCPE (toxic cytopathic effect) and was also an indicator of cell quality.
Diluent control: cells treated with sterile distilled water. This was a negative control for the test articles and assessed any toxic effects of the diluent.

The controls utilized in the virucidal assay are:
Cell only control: cells not infected with virus. This was a negative control for vCPE (viral cytopathic effect) and was also an indicator of cell quality.
Virus only control: cells infected with a 1/1000 dilution of the virus stock. This was a positive control for vCPE.
Diluent control: cells infected with virus that was pre-treated with sterile distilled water for the specified time. This was a negative control for the test articles and assessed any antiviral effects of the diluent.
Antiviral control: cells infected with virus that was pre-treated with citrate buffer at pH3.5. This was a positive control for comparison with the test articles.

The cells of the controls used in each assay were incubated with standard cell infection media.

Cells and Viruses

The cells used in this study were MDCK cells and were supplied from the Retroscreen Virology Ltd. cell bank.

The virus used in this study was Influenza NIBRG-14 (H5N1) virus and was supplied from the Retroscreen Virology Ltd. virus repository. The NIBRG-14 virus is a re-assortant virus between the A-PR8 and Influenza A/Vietnam viruses, created by NIBSC, UK.

The stock titre of Influenza NIBRG-14 virus (AL:870) was approximately 7.0 log₁₀ TCID₅₀/ml.

Before use in the virucidal assay, the stock virus was diluted 10-fold in sterile distilled water. This virus was diluted a further 10-fold when it was added to the test article (section 8.4) to form the reaction mixture. A final 10-fold dilution of the virus was made when the reaction was terminated in standard cell infection media.

Therefore the total dilution of the virus before addition to the plates was 1/1000.

To ensure consistency, the control virus was also diluted 1/1000 before addition to the plate. This was done by diluting the stock virus 100-fold in sterile distilled water, followed by 10-fold in cell infection media. As a result, the virus concentration was reduced from 7 log (or 10⁷) to 4 log (or 10⁴).

Test Variables

The test article was tested for virucidal activity by incubation with the virus for the following time points:
15 seconds
30 seconds
1 minute
5 minutes
   Cytotoxicity Assay

The undiluted test article (100 μl) was added to a monolayer of MDCK cells in quadruplicate and incubated for ˜1 hour at 37° C., 5% CO₂. After incubation, a crystal violet assay was carried out on the cell monolayer. The OD (optical density) readings obtained from this were used to calculate the percentage survivability of the cells.

The crystal violet assay was carried out in accordance to the Retroscreen Virology Ltd. SOP VA024-01.

Virucidal Assay

Virus (40 μl) was added to the undiluted test article (360 μl) and incubated for the specified time points on a platform shaker at room temperature. After incubation, the reaction was terminated by the addition of standard infection media (3.6 ml), which diluted the reaction 10-fold. The terminated mixture was titrated, in quadruplicate, across a 96-well plate of MDCK cells following a 10-fold dilution series. The cells were incubated for ˜1 hour at 37° C., 5% CO₂. After incubation, the supernatant was discarded from the plates and the cell monolayer washed once with PBS (100 μl) and fresh standard infection media (100 μl) added. The cells were incubated for 3 days at 37° C., 5% CO₂. After incubation an HA assay was performed on the supernatant to determine the endpoint of the titrations.

The titrations were carried out in accordance to the Retroscreen Virology Ltd. SOP VA016-04.

The HA assay was carried out in accordance to the Retroscreen Virology Ltd. SOP VAO18-02.

Due to the essential dilutions in the assay, the limit of detection of the assay was 1.5 log₁₀ TCID₅₀/ml.

MDCK Cell Survivability

The survivability of MDCK cells after treatment for ˜1 hour with was >100% (the actual value was calculated to be 197.5%). The measurement obtained was unusually higher than the untreated cell control and may have been a result of the test article (i.e., the test article may promote cell growth).

Reduction in NIBRG-14 (H5N1) Virus Titre

The results of the virucidal assay are indicated in Table 2 below.

TABLE 2
The reduction in virus titre of NIBRG-14 (H5N1) virus
after treatment with the composition for different incubation times
and with the positive control article

| | Virus titre recovered | | Reduction in virus titre | |
|---|---|---|---|---|
| | (log₁₀ TCID₅₀/ml) | | (−log₁₀ | |
| Treatment | Virus control | Test article | TCID₅₀/ml) | (%) |
| Composition/ | 4.50 | 2.50 | 2.00 | 99.00 |
| 15 seconds | | | | |
| Composition/ | 4.50 | 2.50 | 2.00 | 99.00 |
| 30 seconds | | | | |
| Composition/1 | 5.00 | 2.75 | 2.25 | 99.44 |
| minute | | | | |
| Composition/5 | 4.75 | 2.50 | 2.25 | 99.44 |
| minutes | | | | |
| Citrate buffer | 5.25 | <1.50 | >3.75 | >99.98 |
| (pH 3.5) | | | | |

A 2 log reduction means that the virus count was reduced from 10^{4.5} to 10², or from approximately 30,000 to 300. Alternatively expressed, a 2 log reduction means 99% were killed or deactivated.

pH Measurements

The undiluted composition was tested for pH, which was found to be pH3.93.

The results of the cytotoxicity assay are indicative of the test article being non-toxic to the MDCK cell line.

At all time points tested, the test article reduced the virus titre by at least 2 −log₁₀ TCID₅₀/ml (99%). The higher test article incubation time points (1 and 5 minutes) were observed to reduce the virus titre by 0.25 log₁₀ TCID₅₀/ml more than the lower time points (15 and 30 seconds).

It would be of interest to further investigate this test article using different variables (i.e. reducing the test concentration) so as to determine the antiviral range of the test article.

### EXAMPLE 3

This example uses an in-vitro time-kill method to evaluate the antibacterial properties of the composition of Table 1 when challenged with suspensions of 13 different bacterial strains. The composition is evaluated at a 99% v/v concentration.

The percent and log₁₀ reductions from the initial population of each challenge strain will be determined following exposure to the product for 15 seconds, 30 seconds, and one minute. The agar-plating is performed in duplicate.

Insert A

Neutralization studies (SOP L-2007) of the composition were performed using Escherichia coli (ATCC #11229) and Staphylococcus aureus (ATCC #6538) to ensure that the neutralizing solution employed (BBP++) is effective in neutralizing the antimicrobial properties of the product. This neutralization procedure follows guidelines set forth in ASTM E 1054-02, Standard Test Methods for Evaluation of Inactivators of Antimicrobial Agents, except that the challenge suspensions were added to the neutralizing solution prior to the addition of the test product.

Inoculum Preparation

Approximately forty-eight (48) hours prior to testing, separate sterile tubes containing Tryptic Soy Broth were inoculated with the challenge bacteria from lyophilized vials or cryogenic stock cultures containing each species. The broth cultures were incubated at the appropriate temperatures for approximately twenty-four (24) hours, or until sufficient growth was observed.

Appropriately twenty-four (24) hours prior to testing, broth cultures were used to inoculate the surface Tryptic Soy Agar contained in Petri plates. These plates were incubated appropriately until sufficient growth was observed. This will produce lawns of the bacteria on the surface of the agar plates, and colonies from these were used to prepare the challenge suspensions.

Challenge Suspension

Immediately prior to initiating the test procedure, a suspension of each challenge strain was prepared in sterile 0.9% Sodium Chloride Irrigation, USP, by suspending the bacteria from the solid media to achieve challenge suspension concentrations of approximately 1×10⁹ CFU/mL.

Initial Population Determinations

The initial population of each challenge suspension was determined by preparing ten-fold dilutions (e.g., 10⁻¹, 10⁻², 10⁻³, 10⁻⁴, 10⁻⁵, 10⁻⁶, and 10⁻⁷) of each bacterial strain of Butterfield's Phosphate Buffer solution with product neutralizers. Using Tryptic Soy Agar with product neutralizers, pour-plates were prepared, in duplicate, from the inoculum dilutions by plating 0.1 mL of the final dilutions, e.g., 10⁻⁵, 10⁻⁶ and 10⁻⁷, to achieve plate dilution of, e.g., 10⁻⁶, 10⁻⁷, and 10⁻⁸. The plates were incubated at the appropriate temperatures for forty-eight (48) to seventy-two (72) hours, or until sufficient growth was observed.

Testing Procedure

A 0.1 mL aliquot of a challenge suspension containing approximately 1×10⁹ CFU/mL was transferred to a sterile test tube containing 9.9 mL of test product and mixed thoroughly using a vortex mixer and positive displacement pipetter, achieving a 99% (v/v) concentration of the test product.

Each challenge bacterial strain was exposed to the test product for fifteen (15) seconds, thirty (30) seconds, and one (1) minute, timed using a calibrated minute/second timer.

After each exposure time had elapsed, 1.0 mL was removed from each tube containing product/challenge suspension, placed in separate sterile test tubes containing 9.0 mL of Butterfield's Phosphate Buffer solution with product neutralizers (10⁻³ dilution), and mixed thoroughly using a vortex mixer. Appropriate ten-fold dilutions (e.g., 10⁻⁴, 10⁻⁵, and 10⁻⁶) were prepared in Butterfield's Phosphate Buffer solution with product neutralizers, mixing thoroughly using a vortex mixer between dilutions.

From the final dilutions of the product/neutralizer/challenge suspension, 0.1 or 1.0 mL aliquots were pour-plated, in duplicate, using Tryptic Soy Agar with product neutralizers, producing final plated dilutions of, e.g., 10⁻³, 10⁻⁴, 10⁻⁵, 10⁻⁶, and 10⁻⁷. The plates were incubated at the appropriate temperatures for forty-eight (48) to seventy-two (72) hours, or until sufficient growth is observed.

Data Collection

Following incubation, the colonies on the plates were counted manually using a hand-tally counter. Counts in the thirty (30) to three-hundred (300) CFU range were used preferentially in the data calculations. If no counts in the thirty (30) to three-hundred (300) CFU range are observed, those plates with colony counts closest to that range were used for the data calculations.

Calculations

The Log₁₀ Average and the CFU/mL of the average of the duplication plate counts for the initial population of each challenge species and the population after each timed exposure to the test product were calculated as follows:
Log₁₀ Average=Log₁₀(Cᵢ10^{-D})
CFU/mL=(Cᵢ×10^{-D})

Where:
Cᵢ=Average of the Two (2) Plates Counted
D=Dilution Factor of the Plates Counted

The Log₁₀ Reductions of each challenge strain attributable to the test product were calculated for each timed exposure as follows:
Log₁₀ Reduction=Log₁₀ IP−Log₁₀ P_{EX}

Where:
IP=Initial Population of Challenge Strain (CFU/mL)
P_{EX}=Average Population after Exposure to Product (CFU/mL)

The Percent Reductions of each challenge strain attributable to the test product will be calculated for each timed exposure as follows:

$$Percent Reduction = \frac{IP - {P}_{EX}}{IP} \times 100$$

The results of the tests are reported below.

| | | | | Post-Exposure | | |
|---|---|---|---|---|---|---|
| | Microorganism Species | Initial Population | Exposure | Population | Log₁₀ | Percent |
| No. | (ATCC #) | (CFU/mL) | Time | (CFU/mL) | Reduction | Reduction |
| 1 | Enterococcus faecalis | 5.250 × 10⁹ | 15 seconds | 4.5850 × 10⁹ | 0.0589 | 12.6667% |
| | (ATCC #29212) | | 30 seconds | 4.090 × 10⁹ | 0.1085 | 22.0952% |
| | | | 1 minute | 3.470 × 10⁹ | 0.1799 | 33.9048% |
| 2 | Enterococcus faecium | 3.750 × 10⁹ | 15 seconds | 3.0050 × 10⁹ | 0.0962 | 19.8667% |
| | MDR; VRE | | 30 seconds | 2.480 × 10⁹ | 0.1795 | 33.8667% |
| | (ATCC #51559) | | 1 minute | 1.870 × 10⁹ | 0.3022 | 50.1333% |
| 3 | Escherichia coli | 6.650 × 10⁸ | 15 seconds | <1.00 × 10³ | 5.8228 | 99.9998% |
| | (ATCC #11229) | | 30 seconds | <1.00 × 10³ | 5.8228 | 99.9998% |
| | | | 1 minute | <1.00 × 10³ | 5.8228 | 99.9998% |
| 4 | Escherichia coli | 1.0750 × 10⁹ | 15 seconds | <1.00 × 10³ | 6.0314 | 99.9999% |
| | (ATCC #25922) | | 30 seconds | <1.00 × 10³ | 6.0314 | 99.9999% |
| | | | 1 minute | <1.00 × 10³ | 6.0314 | 99.9999% |
| 5 | Escherichia coli | 1.4250 × 10⁹ | 15 seconds | <1.00 × 10³ | 6.1538 | 99.9999% |
| | serotype O157:H7 | | 30 seconds | <1.00 × 10³ | 6.1538 | 99.9999% |
| | (ATCC #43888) | | 1 minute | <1.00 × 10³ | 6.1538 | 99.9999% |
| 6 | Micrococcus luteus | 2.2450 × 10⁸ | 15 seconds | 2.5750 × 10⁸ | 0.0000 | 0.0000% |
| | (ATCC #7468) | | 30 seconds | 6.30 × 10⁶ | 1.5519 | 97.1938% |
| | | | 1 minute | <1.00 × 10⁵ | 3.3512 | 99.9555% |
| 7 | Pseudomonas aeruginosa | 1.730 × 10⁹ | 15 seconds | <1.00 × 10³ | 6.2380 | 99.9999% |
| | (ATCC #15442) | | 30 seconds | <1.00 × 10³ | 6.2380 | 99.9999% |
| | | | 1 minute | <1.00 × 10³ | 6.2380 | 99.9999% |
| 8 | Pseudomonas aeruginosa | 8.050 × 10⁸ | 15 seconds | <1.00 × 10³ | 5.9058 | 99.9999% |
| | (ATCC #27853) | | 30 seconds | <1.00 × 10³ | 5.9058 | 99.9999% |
| | | | 1 minute | <1.00 × 10³ | 5.9058 | 99.9999% |
| 9 | Serratia marcescens | 1.270 × 10⁹ | 15 seconds | 5.350 × 10⁸ | 0.3754 | 57.8740% |
| | (ATCC #14756) | | 30 seconds | 1.1150 × 10⁸ | 1.0565 | 91.2205% |
| | | | 1 minute | 4.250 × 10⁷ | 1.4754 | 96.6535% |
| 10 | Staphylococcus aureus | 1.5250 × 10⁹ | 15 seconds | 4.80 × 10⁸ | 0.5021 | 68.5246% |
| | (ATCC #6538) | | 30 seconds | 2.4550 × 10⁸ | 0.7932 | 83.9016% |
| | | | 1 minute | 8.850 × 10⁷ | 1.2364 | 94.1967% |
| 11 | Staphylococcus aureus | 1.940 × 10⁹ | 15 seconds | 1.00 × 10⁹ | 0.2878 | 48.4536% |
| | (ATCC #29213) | | 30 seconds | 3.80 × 10⁸ | 0.7080 | 80.4124% |
| | | | 1 minute | 1.430 × 10⁸ | 1.1325 | 92.6289% |
| 12 | Staphylococcus aureus | 3.050 × 10⁹ | 15 seconds | 2.7050 × 10⁹ | 0.0521 | 11.3115% |
| | MRSA | | 30 seconds | 1.3350 × 10⁹ | 0.3588 | 56.2295% |
| | (ATCC #33591) | | 1 minute | 6.40 × 10⁸ | 0.6781 | 79.0164% |
| 13 | Staphylococcus | 1.290 × 10⁹ | 15 seconds | 8.00 × 10⁷ | 1.2075 | 93.7984% |
| | epidermidis | | 30 seconds | 1.50 × 10⁷ | 1.9345 | 98.8372% |
| | (ATCC #12228) | | 1 minute | 9.50 × 10⁵ | 3.1329 | 99.9264% |

The above specification, examples and data provide a complete description of the manufacture and use of the composition of the invention. Since many embodiments of the invention can be made without departing from the spirit and scope of the invention, the invention resides in the claims hereinafter appended.

We claim:

1. A skin disinfectant composition comprising:
(a) an effective amount of a disinfectant active component to provide disinfectant properties to skin tissue;
(b) an effective amount of a skin bonding polymer component to hold the disinfectant active component to skin tissue so that the disinfectant active component becomes available on skin tissue to provide disinfectant properties; and
(c) water.

2. A skin disinfectant composition according to claim 1, wherein the disinfectant active component comprises a chlorhexidine salt having a water solubility of less than about 1% wt/vol in water at 20° C.

3. A skin disinfectant composition according to claim 2, wherein the chlorhexidine salt comprises a salt of chlorhexidine and fatty acid having a fatty chain containing at least 8 carbon atoms.

4. A skin disinfectant composition according to claim 2, wherein the chlorhexidine salt comprises a salt of chlorhexidine and polycarboxylic acid.

5. A skin disinfectant composition according to claim 2, wherein the chlorhexidine salt comprises a salt of chlorhexidine and succinic acid

6. A skin disinfectant composition according to claim 1, wherein the composition comprises about 0.1 wt. % to about 5 wt. % of the disinfectant active component.

7. A skin disinfectant composition according to claim 1, wherein in the composition comprises about 1.25 wt. % to about 4 wt. % of the disinfectant active component.

8. A skin disinfectant composition according to claim 2, wherein in the chlorhexidine salt has a water solubility of less than about 0.5% wt/vol in water at 20° C.

9. A skin disinfectant composition according to claim 1, wherein in the skin bonding polymer component comprises a hydrophobic polymer/hydrophilic polymer adduct.

10. A skin disinfectant composition according to claim 9, wherein in the hydrophobic polymer/hydrophilic polymer adduct comprises a poly (vinylpyrrolidone-alkylene)polymer and a polymer comprising repeating carboxylic acid groups hydroxyl groups, or a mixture of carboxylic acid groups and hydroxyl groups.

11. A skin disinfectant composition according to claim 10, wherein the poly (vinylpyrrolidone-alkylene)polymer comprises a polymer having an alkylene group containing about 10 carbon atoms to about 30 carbon atoms.

12. A skin disinfectant composition according to claim 1, wherein the skin bonding polymer component has a ratio of pyrrolidone groups to a combination of carboxylic acid groups and hydroxyl groups of about 1:1 to about 5:1.

13. A skin disinfectant composition according to claim 1, wherein the composition comprises at least about 50 wt. % water.

14. A skin disinfectant composition according to claim 1, wherein the composition comprises about 0.5 wt. % to about 6 wt. % of a surfactant.

15. A skin disinfectant composition according to claim 1, wherein the composition comprises about 0.1 wt. % to about 2 wt. % of a thickener.

16. A skin disinfectant composition according to claim 1, wherein the composition comprises about 0.5 wt. % to about 5 wt. % of a moisturizer.

17. A skin disinfectant composition according to claim 1, wherein the composition, after application skin tissue, provides disinfectant properties for at least 2 hours after application of the composition to the skin tissue, wherein the skin tissue is washed every half hour for 30 seconds with a mild soap.

18. A skin disinfectant composition according to claim 1, wherein the composition, after application skin tissue, provides disinfectant properties for at least 4 hours after application of the composition to the skin tissue, wherein the skin tissue is washed every half hour for 30 seconds with a mild soap.

19. A method for using a skin disinfectant composition comprising:
Applying the skin disinfectant composition to skin tissue, wherein the skin disinfectant composition comprises:
(a) an effective amount of a disinfectant active component to provide disinfectant properties to skin tissue;
(b) an effective amount of a skin bonding polymer component to hold the disinfectant active component to skin tissue so that the disinfectant active component becomes available on skin tissue to provide disinfectant properties; and
(c) water.

20. A method according to claim 19, wherein the skin tissue comprises skin tissue provided on a person's hands.
